(19) United States
(12) Patent Application Publication
(10) Pub. No.: US 2021/0077637 A1
(43) Pub. Date: Mar. 18, 2021

(54) PH and Oxygen dual-sensitive magnetic resonance imaging contrast agent and preparation method thereof

(71) Applicant: Harbin Medical University, Harbin City (CN)

(72) Inventors: Xinlin Sun, Harbin (CN); Lina Wu, Harbin (CN); Rong A, Harbin (CN); OLAGBAJU OLUWATOSIN ATINUKE, harbin (CN); Kai Wang, Harbin (CN); Lili Yang, Harbin (CN); Lixin Cheng, Harbin (CN); Wenju Qiao, Harbin (CN); Xiaohong Sun, Harbin (CN)

(21) Appl. No.: 17/060,059

(22) Filed: Sep. 30, 2020

(30) Foreign Application Priority Data
Dec. 23, 2019 (CN) 201911335620X

(51) Int. Cl. A61K 49/18; A61K 49/08; A61K 49/10

(52) U.S. Cl. CPC A61K 49/1812; B82Y 5/00; A61K 49/10; A61K 49/085

(57) ABSTRACT

The present disclosure provides a pH and oxygen dual-sensitive magnetic resonance imaging contrast agent and a preparation method thereof, which is a dual-modal nanoparticle contrast agent with ¹⁹F signal and CEST dual signal (¹⁹F-CEST), a CEST contrast agent that is dual-sensitive to pH and oxygen. The preparation method comprises: mixing a variety of phospholipid surfactants and cholesterol well to obtain a blend of phospholipid surfactants, which is dissolved in chloroform or a mixed solvent of chloroform and methanol, evaporated to dryness with a rotary evaporator and dried overnight in a vacuum oven at 40° C. after adding rhodamine, finally, it is dispersed in water containing glycerin by mechanical dispersion or ultrasonic vibration to obtain a lipid modifier; mixing perfluorocarbon, the lipid modifier obtained in step (1), glycerin, and water and ultrasonically mixing well with a probe, and extruding with an extruder to prepare a perfluorocarbon nanoemulsion.

### CROSS REFERENCE TO RELATED APPLICATION

This application claims priority of Chinese Application No. CN CN201911335620X filed on 2019 Dec. 23 and entitled “pH and oxygen dual-sensitive magnetic resonance imaging contrast agent and preparation method thereof”.

### TECHNICAL FIELD

The invention belongs to the technical field of magnetic resonance imaging, in particular to a method for preparing a pH and oxygen dual-sensitive magnetic resonance imaging contrast agent, and the dual-sensitive contrast agent obtained therefrom.

### BACKGROUND

Pathological tissues (e.g., inflammations, infections, tumor tissues) are significantly different from normal tissues, such as pH, temperature, and surrounding environment.

The basic principle of chemical exchange saturation transfer (CEST) imaging is to apply a pre-saturation pulse (Radio Frequency, RF) to the exchangeable proton pool to saturate the hydrogen protons in the exchangeable pool, and the saturated hydrogen protons then chemically exchange with the hydrogen protons in the surrounding free pool to reduce the water signal. Therefore, by detecting the changes in the water molecule signal, the information of biological macromolecules such as concentration and metabolism can be reflected indirectly. Because the concentration of macromolecular solutes or metabolites in the organism is generally small (micromolar or millimolar), it is difficult to observe the signal on conventional MR images, and the chemical exchange saturation transfer actually plays a role in signal amplification, making information about low concentrations of solutes or metabolites can be detected.

In general, CEST imaging can be used to non-invasively detect the pH of the region of interest in living tissues, especially the measurement of the pH outside the tumor cells.

Since many biological endogenous macromolecules (such as protein, glucose, inositol, and glutamate) contain active hydrogen protons and meet the basic characteristics of CEST contrast agents, they can be used as endogenous CEST contrast agents. (Chemical Exchange Saturation Transfer (CEST) Imaging: Description of Technique and Potential Clinical Application, Curr Radiol Rep. 2013 Jun. 1; 1 (2): 102-114)

Perfluorocarbon (PFC) has high oxygen solubility and hydrophobicity, and its safety and biocompatibility have been fully confirmed. However, the current clinically applied 1H-MRI magnetic field strength does not exceed 3.0 T, and 19F-MRI requires a higher magnetic field strength (4.7˜14.0 T) to compensate for its relatively low sensitivity, in addition the dilution effect of circulating blood has brought some obstacles to the research of perfluorocarbon (PFC) targeting probes. (Perfluorooctylbromide Nanoparticles for Ultrasound Imaging and Drug Delivery, Int J Nanomedicine. 2018V13N:3053-3067; Eight-Coordinate, Stable Fe(II) Complex as a Dual ¹⁹F and CEST Contrast Agent for Ratiometric pH Imaging, Inorg Chem. 2017 Oct. 16; 56(20):12206-12213)

Tumor extracellular pH (pHe) is usually lower than tumor intracellular pH (pHi), which is the opposite of normal tissues. However, this tumor extracellular acidic environment promotes cancer progression by promoting tumor cell proliferation, evading apoptosis, metabolic adaptation, migration, and invasion. Therefore, the measurement of tumor extracellular pH is very important for the analysis of the degree of malignancy. However, the body composition is complex, and there are many interference factors to the magnetic field, such as direct water saturation (DS) and conventional MT contrast (MTC), nuclear overhauser effect (NOE); and the sensitivity of endogenous CEST contrast agent is not high and easily affected by the mixing effect, and it is difficult to achieve accurate determination of the target compound in vivo imaging. (Potentiometric and Relaxometric Properties of a Gadolinium-Based MRI Contrast Agent for Sensing Tissue pH, Inorg Chem. 2007, 46, 5260-5270; A General MRI-CEST Ratiometric Approach for pH Imaging: Demonstration of in Vivo pH Mapping with Iobitridol, J Am Chem Soc. 2014V136N41:14333-6).

The metabolism, blood oxygen saturation, and tissue oxygen content of tumor tissues are also different from normal tissues, therefore, evaluation of tumor blood vessels and blood oxygen saturation by BOLD-MRI, and imaging based on changes in hemoglobin oxygen saturation level are receiving increasing attention. (Imaging tumour hypoxia with oxygen-enhanced MRI and BOLD MRI, Br J Radiol. 2019V92N1095:20180642)

### SUMMARY

The present invention provides a pH and oxygen dual-sensitive magnetic resonance imaging contrast agent and a preparation method and use thereof, which is a dual-modal nanoparticle contrast agent with ¹⁹F signal and CEST dual signal (¹⁹F-CEST), a CEST contrast agent that is dual-sensitive to pH and oxygen, and can be used to detect the characteristics of very few substances by CEST imaging to make up for the low sensitivity of ¹⁹F signal.

The present invention provides a method for preparing a pH and oxygen double-sensitive magnetic resonance imaging contrast agent, which comprises the following steps:

(1) preparation of lipid modifier: mixing a variety of phospholipid surfactants and cholesterol well to obtain a blend of phospholipid surfactants, which is dissolved in chloroform or a mixed solvent of chloroform and methanol, evaporated to dryness with a rotary evaporator and dried overnight in a vacuum oven at 40° C. after adding rhodamine, finally, it is dispersed in water containing glycerin by mechanical dispersion or ultrasonic vibration to obtain a lipid modifier;

the phospholipid surfactant blend is composed of phosphatidylcholine liposomes, phosphatidylglycerol liposomes and cholesterol;

(1) preparation of perfluorocarbon nanoemulsion: mixing perfluorocarbon, the lipid modifier obtained in step (1), glycerin, and water and ultrasonically mixing well with a probe, and extruding with an extruder to prepare a perfluorocarbon nanoemulsion.

To prepare a CEST contrast agent sensitive to pH and oxygen, further, in the lipid modifier obtained in step (1), the mass ratio of the blend of phospholipid surfactant, the mixed solvent of chloroform and methanol, and rhodamine is (80-95):(35-45):(0.5-2).

Further, in the lipid modifier obtained in step (1), the mass ratio of the blend of phospholipid surfactant, the mixed solvent of chloroform and methanol, and rhodamine is (80-90):(35-45):(1.0-2).

In step (1), the phosphatidylcholine liposome includes: dimyristoyl phosphatidylcholine (DMPC), dilauroyl phosphatidylcholine (DLPC), dipalmitoyl phosphatidylcholine (DPPC)), dioleoyl phosphatidylcholine (DOPC), distearoyl phosphatidylcholine (DSPC), diarachido phosphatidylcholine (DAPC) and palmitoyloleyl phosphatidylcholine (POPC);

the phosphatidylglycerol liposome includes: dipalmitoyl phosphatidylglycerol (DPPG).

Further, the blend of phospholipid surfactant is composed of dipalmitoyl phosphatidylcholine (DPPC), dipalmitoyl phosphatidylglycerol (DPPG), and cholesterol.

Further, the molar ratio of phosphatidylcholine liposome, phosphatidylglycerol liposome and cholesterol is (60-80):(10-15):(10-25).

Further, the perfluorocarbon nanoemulsion obtained in step (2), perfluorocarbon and glycerin account for 10-40% of the total mass, water accounts for 55-85% of the total mass, and phospholipid surfactants account for 1-5% of the total mass.

Further, the perfluorocarbon nanoemulsion obtained in step (2), perfluorocarbon and glycerin account for 20-40% of the total mass, water accounts for 55-75% of the total mass, and phospholipid surfactants account for 1-5% of the total mass.

Further, the perfluorocarbon nanoemulsion obtained in step (2), the mass ratio of perfluorocarbon to glycerin 10-20:1.

Further, the the perfluorocarbon nanoemulsion obtained in step (2), the mass ratio of perfluorocarbon to glycerin is 15-20:1, or 15-18:1, or 16-18:1.

Further, the perfluorocarbon (PFC) is selected from one or more of brominated perfluorooctane, perfluoro-15-crown ether-5, FC-3280 ((C8F18)) and FC-77 ((C8F160)).

Further, the ultrasonic power of ultrasonic oscillation in step (1) is smaller than the ultrasonic power of ultrasonic processing in step (2).

In step (1), the ultrasonic oscillation time is 5 seconds to 10 seconds, and the power P₁ is 400 W>P1>300 W; in step (2), the ultrasonic treatment time is 60 seconds-70 seconds, and the power P2 is: P2>P1 and 450 W>P2≥400 W.

Further, the power P₁ is 380 W>P1>350 W; and the power P2 is 440 W≥P2≥400 W.

The present invention also provides a pH and oxygen dual-sensitive magnetic resonance imaging contrast agent prepared by any one of the above methods. Dialysis is used to remove components that are not effectively coated in the emulsion obtained in step (2) to obtain a CEST contrast agent probe.

### Beneficial Effects

CEST imaging can be used to non-invasively detect the pH of the region of interest in living tissues, especially the measurement of the pH outside the tumor cells. However, the body composition is complex, and there are many interference factors to the magnetic field, such as DS effect, MT effect and NOE effect; and the sensitivity of endogenous CEST contrast agent is not high and easily affected by the mixing effect, and it is difficult to achieve accurate determination of the target compound during imaging. The metabolism, blood oxygen saturation, and tissue oxygen content of tumor tissues are also different from normal tissues, therefore, evaluation of tumor blood vessels and blood oxygen saturation by BOLD-MRI, and imaging based on changes in hemoglobin oxygen saturation level are receiving increasing attention.

The inventors of the present invention have discovered for the first time the effect and law of oxygen on the signal of perfluorocarbon (PFC) nanoemulsion, which can lay a research foundation for the oxygen carrying and release degree of biological perfluorocarbon (PFC) nanoemulsion. By discovering and studying the oxygen carrying properties of perfluorocarbon (PFC) and the effect of oxygen content on the CEST signal, thus it is possible to analyze the oxygen carrying amount of the probes of tissues or tumor sites over time by CFC imaging using perfluorocarbon (PFC) nanoparticles, and a CEST contrast agent sensitive to pH and oxygen is prepared according to this.

### BRIEF DESCRIPTION OF THE DRAWINGS

FIG. 1. Hydrated particle size diagram of the pH and oxygen dual-sensitive ¹⁹F-MRI/CEST multimodal imaging nanoparticles.

FIG. 2. ZETA potential diagram of the pH and oxygen dual-sensitive ¹⁹F-MRI/CEST multimodal imaging nanoparticles.

FIG. 3. FIG. 3-1 is the ¹H magnetic resonance spectrum of the pH and oxygen dual-sensitive ¹⁹F-MRI/CEST multimodal imaging nanoparticles; FIG. 3-2 is an enlarged view of A shown in FIG. 3-1.

FIG. 4. ¹H-MR CEST imaging Z-spectrum of the pH and oxygen dual-sensitive ¹⁹F-MRI/CEST multimodal imaging nanoparticles.

FIG. 5. Transmission electron microscopy (TEM) image of the pH and oxygen dual-sensitive ¹⁹F-MRI/CEST multimodal imaging nanoparticles.

FIG. 6. T1 weighting (6-1) and ¹⁹F signal image (6-2) of the pH and oxygen dual-sensitive ¹⁹F-MRI/CEST multimodal imaging nanoparticles. The volume dilution ratios of the pH and oxygen dual-sensitive ¹⁹F-MRI/CEST multimodal imaging nanoparticles. are: 1, 1:5, 1:10, 1:20, 1:100, respectively.

FIG. 7. CEST signal ST%map color map of the pH and oxygen dual-sensitive ¹⁹F-MRI/CEST multimodal imaging nanoparticles. The pre-saturation pulse power in FIG. (7-1) is 1.2 μT and the saturation time is 3 s, the pre-saturation pulse power in FIG. (7-2) is 2.4 μT and the saturation time is 3 s. The volume dilution ratio of the pH and oxygen dual-sensitive 19F-MRI/CEST multimodal imaging nanoparticles. are: 1, 1:5, 1:10, 1:20, 1:100, respectively)

FIG. 8. Histograms of the pH and oxygen dual-sensitive ¹⁹F-MRI/CEST multimodal imaging nanoparticles with different dilution ratios between different pre-saturation pulses and different saturation time. The pre-saturation pulse power in FIG. (8-1) is 1.2 μT, and the pre-saturation pulse power in FIG. (8-2) is 2.4 μT.

FIG. 9. pH sensitive standard curve of the pH and oxygen dual-sensitive ¹⁹F-MRI/CEST multimodal imaging nanoparticles.

FIG. 10. O₂ sensitive standard curve of the pH and oxygen dual-sensitive ¹⁹F-MRI/CEST multimodal imaging nanoparticles.

### DETAILED DESCRIPTION

The technical solutions in the embodiments of the present invention will be described clearly and completely in conjunction with the drawings in the embodiments of the present invention. Obviously, the embodiments described are only a part of the embodiments of the present invention, but not all the embodiments. Based on the embodiments of the present invention, all other embodiments obtained by those of ordinary skill in the art without making creative efforts are all in the protection scope of the present invention.

### Embodiment 1

A pH and oxygen double-sensitive magnetic resonance imaging contrast agent is prepared by the method as follows:

(1) preparation of lipid modifier: mixing a variety of phospholipid surfactants and cholesterol well to obtain a blend of phospholipid surfactants, which is dissolved in a mixed solvent of chloroform and methanol, evaporated to dryness with a rotary evaporator and dried overnight in a vacuum oven at 40° C. after adding rhodamine, finally, it is dispersed in water containing glycerin by ultrasonic vibration to obtain a lipid modifier.

The blend of phospholipid surfactant is composed of dipalmitoyl phosphatidylcholine (DPPC), dipalmitoyl phosphatidylglycerol (DPPG), and cholesterol, and the molar ratio between them is DPPC:DPPG:cholesterol=75:15:20.

The mass ratio of the blend of phospholipid surfactant, the mixed solvent of chloroform and methanol, and rhodamine is 85:35:0.7.

The ultrasonic vibration processing time is 5 seconds, and power is 380 W.

(2) preparation of perfluorocarbon nanoemulsion: mixing perfluorocarbon, the lipid modifier obtained in step (1), glycerin, and water and ultrasonically mixing well with a probe, and extruding with an extruder (Avanti mini extruder) to prepare a perfluorocarbon nanoemulsion.

Perfluorocarbon accounts for 31.25% of the total mass, glycerin accounts for 2% of the total mass, water accounts for 64.75% of the total mass, and phospholipid surfactants account for 2% of the total mass.

Perfluorocarbon (PFC) is selected from perfluoro-15-crown ether-5.

The ultrasonic processing time is 65 seconds, and power is 400 W.

Dialysis is used to remove components that are not effectively coated in the emulsion in step (2) to obtain a CEST contrast agent probe.

### Embodiment 2

A pH and oxygen double-sensitive magnetic resonance imaging contrast agent is prepared by the method as follows:

(1) preparation of lipid modifier: mixing a variety of phospholipid surfactants and cholesterol well to obtain a blend of phospholipid surfactants, which is dissolved in a mixed solvent of chloroform and methanol, evaporated to dryness with a rotary evaporator and dried overnight in a vacuum oven at 40° C. after adding rhodamine, finally, it is dispersed in water containing glycerin by ultrasonic vibration to obtain a lipid modifier.

The blend of phospholipid surfactant is composed of dipalmitoyl phosphatidylcholine (DPPC), dipalmitoyl phosphatidylglycerol (DPPG), and cholesterol, and the molar ratio between them is DPPC:DPPG:cholesterol=75:15:20.

The mass ratio of the blend of phospholipid surfactant, the mixed solvent of chloroform and methanol, and rhodamine is 90:35:1.5.

The ultrasonic vibration processing time is 8 seconds, and power is 360 W.

(2) preparation of perfluorocarbon nanoemulsion: mixing perfluorocarbon, the lipid modifier obtained in step (1), glycerin, and water and ultrasonically mixing well with a probe, and extruding with an extruder (Avanti mini extruder) to prepare a perfluorocarbon nanoemulsion.

Perfluorocarbon accounts for 31.25% of the total mass, glycerin accounts for 2% of the total mass, water accounts for 64.75% of the total mass, and phospholipid surfactants account for 2% of the total mass.

Perfluorocarbon (PFC) is selected from perfluoro-15-crown ether-5.

The ultrasonic processing time is 70 seconds, and power is 430 W.

Dialysis is used to remove components that are not effectively coated in the emulsion in step (2) to obtain a CEST contrast agent probe.

### Embodiment 3

A pH and oxygen double-sensitive magnetic resonance imaging contrast agent is prepared by the method as follows:

(1) preparation of lipid modifier: mixing a variety of phospholipid surfactants and cholesterol well to obtain a blend of phospholipid surfactants, which is dissolved in a mixed solvent of chloroform and methanol, evaporated to dryness with a rotary evaporator and dried overnight in a vacuum oven at 40° C. after adding rhodamine, finally, it is dispersed in water containing glycerin by ultrasonic vibration to obtain a lipid modifier.

The blend of phospholipid surfactant is composed of dipalmitoyl phosphatidylcholine (DPPC), dipalmitoyl phosphatidylglycerol (DPPG), and cholesterol, and the molar ratio between them is DPPC:DPPG:cholesterol=75:15:20.

The mass ratio of the blend of phospholipid surfactant, the mixed solvent of chloroform and methanol, and rhodamine is 85:35:2.

The ultrasonic vibration processing time is 5 seconds, and power is 350 W.

(2) preparation of perfluorocarbon nanoemulsion: mixing perfluorocarbon, the lipid modifier obtained in step (1), glycerin, and water and ultrasonically mixing well with a probe, and extruding with an extruder (Avanti mini extruder) to prepare a perfluorocarbon nanoemulsion.

Perfluorocarbon accounts for 36.45% of the total mass, glycerin accounts for 2% of the total mass, water accounts for 62.55% of the total mass, and phospholipid surfactants account for 2% of the total mass.

Perfluorocarbon (PFC) is selected from perfluoro-15-crown ether-5.

The ultrasonic processing time is 60 seconds, and power is 400 W.

Dialysis is used to remove components that are not effectively coated in the emulsion in step (2) to obtain a CEST contrast agent probe.

The characterization and effect verification experiments of the CEST contrast agent probe of the embodiment 1 of the present invention are further described below.

### (1) MR CEST Imaging of the pH and Oxygen Dual-Sensitive ¹⁹F-MRI/CEST Multimodal Imaging Nanoparticles

The synthesized pH and oxygen dual-sensitive ¹⁹F-MRI/CEST multimodal imaging nanoemulsion is the original solution, and is diluted in a 0.25 ml EP tube with a volume ratio of 1, 1/5, 1/10, 1/20, 1/100, respectively, and is fixed it with distilled water in a 40 ml centrifuge tube for ¹⁹FMR, T1RARE, T1mapping, T2mapping, CEST EPI sequence scanning, and the CEST signal efficiency of the probe is analyzed and calculated through data processing by MATLAB and Graphpad Prism7. Scanning parameters: Repetition Time: 10000 ms, Echo Time: 20 ms, Slice thickness: 2 mm, FOV: 35*35 mm, Bandwidth: 300000, Averages: 1, Repetitions: 95, Segments: 1, Number Offset Experiment: 95, Min CEST Offset: 4000, Max CEST Offset: −4000, RF Amplitude μT: 3, Length: 5000 ms, Duration time: 3 s, 5 s, 8 s, Saturation power: 0.2, 0.5, 0.8, 1.0, 1.2, 2.4, 3.5, 4.7 μT.

### (2) Characterization of the pH and Oxygen Dual-Sensitive ¹⁹F-MRI/CEST Multimodal Imaging Nanoparticles

Characterization of the pH and oxygen dual-sensitive ¹⁹F-MRI/CEST multimodal imaging nanoparticles use transmission electron microscopy (TEM) to observe the size, morphology and structural characteristics of nanoparticles; use nanoparticle size potential analyzer to measure the hydrated particle size, Zeta potential and polydispersity index of the pH and oxygen dual-sensitive ¹⁹F-MRI/CEST multimodal imaging nanoparticles at room temperature; the results meet the requirements of nanometer particle size, and the solution has good stability.

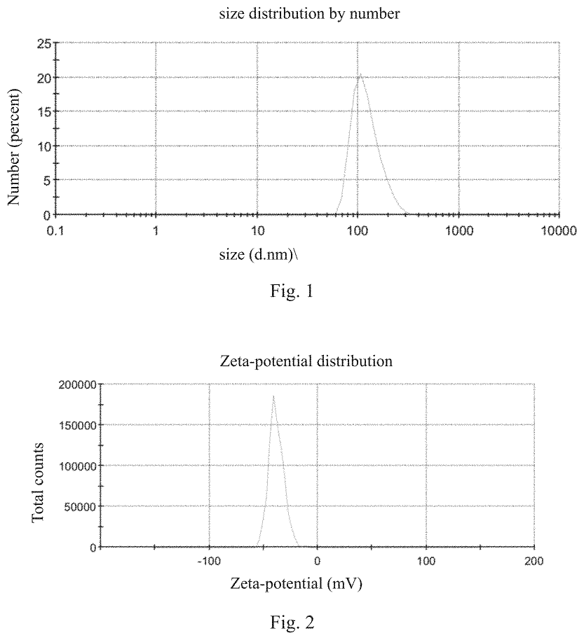

The hydration particle size of the pH and oxygen dual-sensitive ¹⁹F-MRI/CEST multimodal imaging nanoparticles under dynamic light scattering (DLS) is 123.4 nm, which provides a powerful guarantee for effectively improving the EPR of the tumor area (FIG. 1).

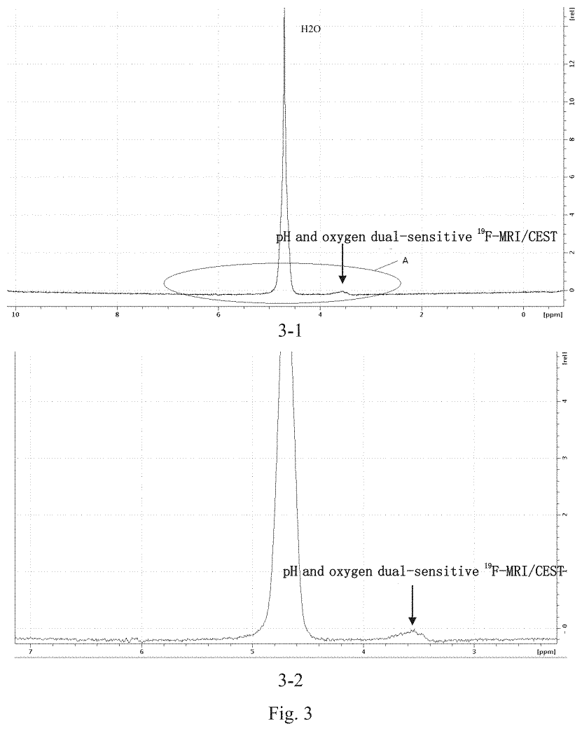

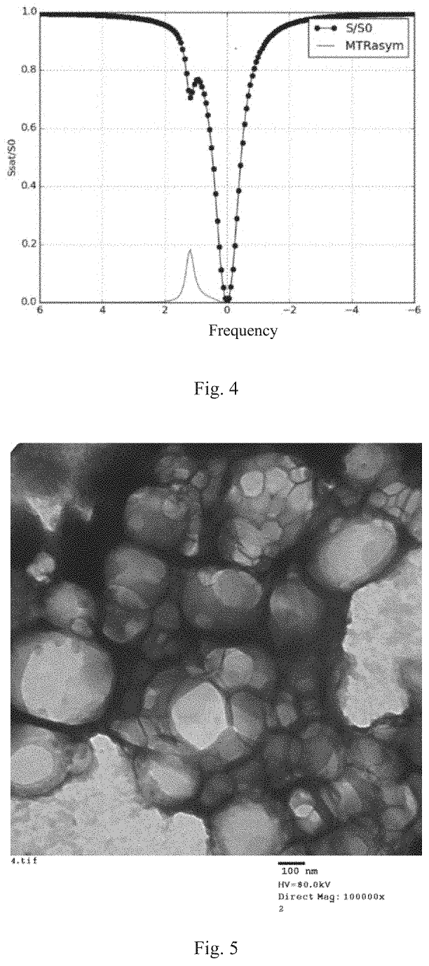

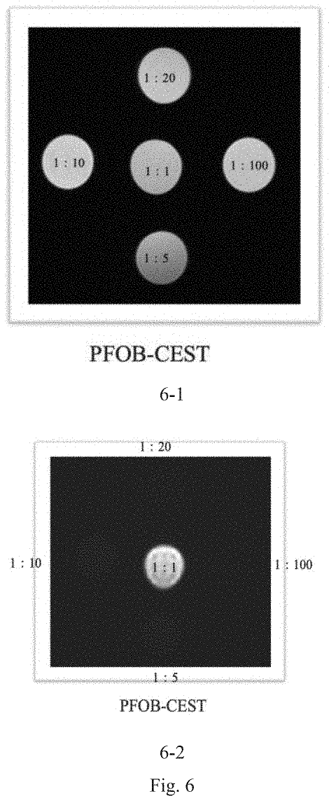

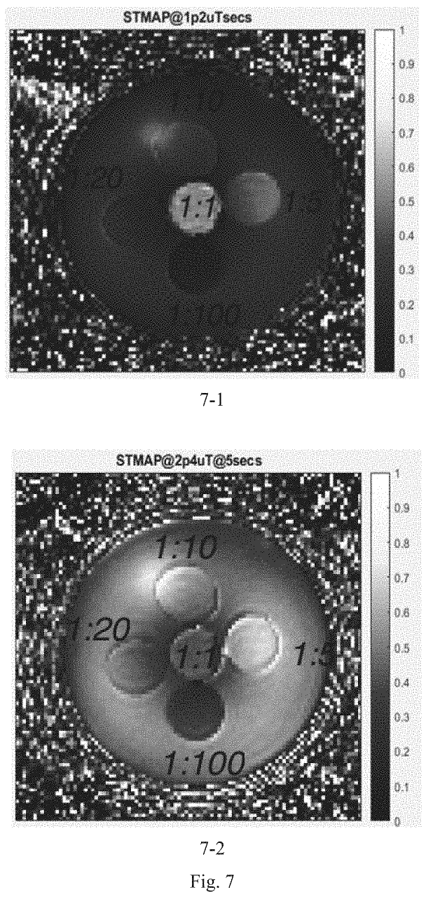

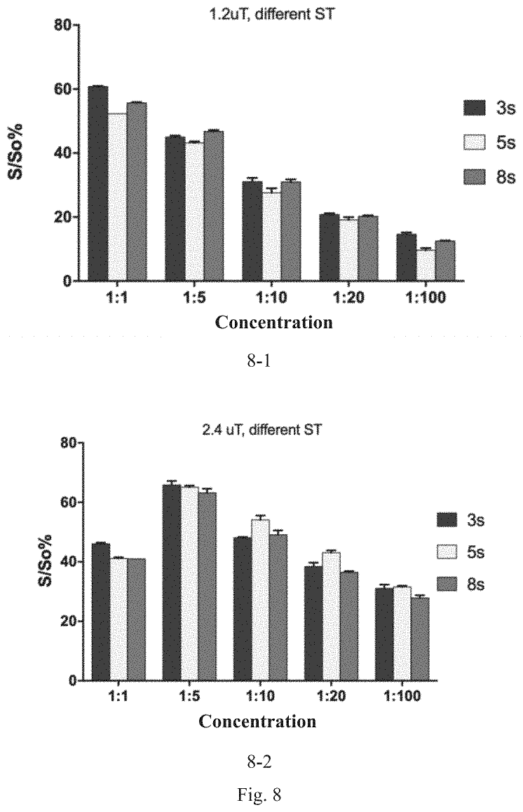

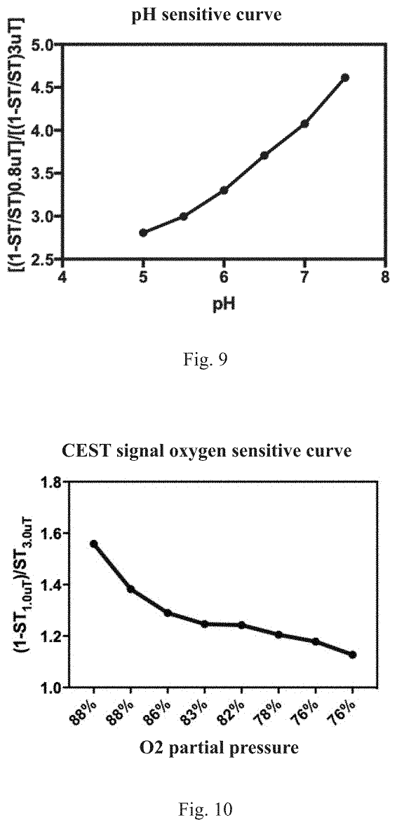

The content of phosphatidylcholine liposomes modifying the cell model of the pH and oxygen dual-sensitive ¹⁹F-MRI/CEST multimodal imaging nanoparticles in the solute is extremely small, and it is difficult to detect the proton spectrum peak on the 1H magnetic resonance spectrum (FIG. 3), as shown in FIG. 3-1, almost no proton spectrum peak of the pH and oxygen dual-sensitive ¹⁹F-MRI/CEST multimodal imaging nanoparticles is detected. After magnifying A shown in FIG. 3-1 at 3-6 ppm (FIG. 3-2), a very small proton spectrum peak can be detected, but under CEST imaging technology, the −OH group CEST signal peak derived from phosphatidylcholine liposomes can be easily found (FIG. 4).

The 19F and CEST dual signal sources of the pH and oxygen dual-sensitive ¹⁹F-MRI/CEST multimodal imaging nanoparticles can effectively improve the quality of spatial positioning. (When the dilution ratio is 1:5, the ¹⁹F signal is significantly weakened on ¹⁹F-MRI, but on ¹H-MR CEST imaging, when the pre-saturation pulse power is 2.4 μT and the saturation time is 3 s, up to 63% of the CEST signal can be detected; when the dilution ratio is 1:100, the ¹⁹F signal is basically undetectable on ¹⁹F-MRI, but up to 36% of the signal can still be detected on CEST imaging, so it can provide a “beacon” effect for 19F signal positioning. FIG. (6-2), FIG. (7-2)).

CEST contrast agent is affected by the dissociation coefficient of acidity of free water. When the pre-saturation pulse is 0.8 μT and 3 μT, the pH-sensitive standard curve of the pH and oxygen dual-sensitive ¹⁹F-MRI/CEST multimodal imaging nanoparticles is obtained by the ratiometer method, which can be further used to detect the acidity of the area of interest.

Because of the oxygen-carrying properties of perfluorocarbon (PFC) nanoemulsions, we have discovered for the first time the effect and law of oxygen on the signal of perfluorocarbon (PFC) nanoemulsion, which can lay a research foundation for the oxygen carrying and release degree of biological perfluorocarbon (PFC) nanoemulsion.

Although the embodiments of the present invention have been shown and described above, it can be understood that the above embodiments are exemplary and cannot be construed as limitations to the present invention, and those of ordinary skill in the art may make changes, modifications, substitutions, and variations to the above-described embodiments within the scope of the present invention without departing from the principle and purpose of the present invention. The scope of the present invention is defined by the appended claims and equivalents thereof.

The above are only specific embodiments of the present invention, but the scope of protection of the present invention is not limited to this. Any person skilled in the art can easily think of changes or substitutions within the technical scope disclosed by the present invention should be covered by the protection scope of the present invention. Therefore, the protection scope of the present invention should be subject to the protection scope of the claims.

What is claimed is:

1. A method for preparing a pH and oxygen dual-sensitive magnetic resonance imaging contrast agent, wherein the method comprises the following steps:
(1) preparation of lipid modifier:
mixing a variety of phospholipid surfactants and cholesterol well to obtain a blend of phospholipid surfactants, which is dissolved in chloroform or a mixed solvent of chloroform and methanol, evaporated to dryness with a rotary evaporator and dried overnight in a vacuum oven at 40° C. after adding rhodamine, finally, dispersing in water containing glycerin by mechanical dispersion or ultrasonic vibration to obtain a lipid modifier;
the phospholipid surfactant blend is composed of phosphatidylcholine liposome, phosphatidylglycerol liposome and cholesterol;
(2) preparation of perfluorocarbon nanoemulsion: mixing perfluorocarbon, the lipid modifier obtained in step (1), glycerin, and water and ultrasonically mixing well with a probe, and extruding with an extruder to prepare a perfluorocarbon nanoemulsion.
(3)

[symbol]

2. The method for preparing a pH and oxygen dual-sensitive magnetic resonance imaging contrast agent according to claim 1, wherein in the perfluorocarbon nanoemulsion obtained in step (2), perfluorocarbon and glycerin account for 10-40% of the total mass, water accounts for 55-85% of the total mass, and phospholipid surfactants account for 1-5% of the total mass.

3. The method for preparing a pH and oxygen dual-sensitive magnetic resonance imaging contrast agent according to claim 2, wherein in the perfluorocarbon nanoemulsion obtained in step (2), the mass ratio of perfluorocarbon to glycerin 10-20:1.

4. The method for preparing a pH and oxygen dual-sensitive magnetic resonance imaging contrast agent according to claim 1, wherein the perfluorocarbon is selected from one or more of brominated perfluorooctane, perfluoro-15-crown ether-5, FC-3280 and FC-77.

5. The method for preparing a pH and oxygen dual-sensitive magnetic resonance imaging contrast agent according to claim 1, wherein in the lipid modifier obtained in step (1), the mass ratio of the blend of phospholipid surfactant, the mixed solvent of chloroform and methanol, and rhodamine is (80-95):(35-45):(0.5-2).

6. The method for preparing a pH and oxygen dual-sensitive magnetic resonance imaging contrast agent according to claim 1, wherein in step (1), the ultrasonic oscillation time is 5 seconds to 10 seconds, and the power P₁ is 400 W>P1>300 W; in step (2), the ultrasonic treatment time is 60 seconds-70 seconds, and the power P2 is: P2>P1 and 450 W>P2≥400 W.

7. The method for preparing a pH and oxygen dual-sensitive magnetic resonance imaging contrast agent according to claim 1, wherein the molar ratio of phosphatidylcholine liposome, phosphatidylglycerol liposome and cholesterol is (60-80):(10-15):(10-25).

8. A pH and oxygen dual-sensitive magnetic resonance imaging contrast agent prepared by the method according to claim 1.
